(19) United States
(12) Patent Application Publication
(10) Pub. No.: US 2023/0272451 A1
(43) Pub. Date: Aug. 31, 2023

(54) SYSTEM AND METHOD FOR DETERMINING ENZYME ACTIVITY IN GRAIN MATERIAL

(71) Applicant: GLYCOSPOT APS, Søborg (DK)

(72) Inventors: Julia SCHÜCKEL, Dresden (DE); Robert BAYER, Søborg (DK); Raimon PARÉS VIADER, Søborg (DK); Lennart HANSEN, Søborg (DK); Stjepan Kresimir KRACUN, Søborg (DK); Roberto Thomas LACENTRA, Søborg (DK); Troels Balmer CHRISTENSEN, Søborg (DK)

(21) Appl. No.: 18/004,136

(22) PCT Filed: Jun. 25, 2021
(86) PCT No.: PCT/DK2021/050209

§ 371 Date: Jan. 3, 2023

(30) Foreign Application Priority Data
Jul. 3, 2020 (DK) PA 2020 00798

(51) Int. Cl. C12Q 1/40; G01N 21/78

(52) U.S. Cl. CPC C12Q 1/40; G01N 21/78

(57) ABSTRACT

A method of determining activity of a target enzyme of a grain material is described. The method comprises providing a fluid extract sample of the grain material using a preselected extraction procedure, providing a dyed and/or chromogenic substrate for the target enzyme, subjecting the fluid extract sample to the substrate for a preselected incubating time, and determining the target enzyme activity of the grain material. The extraction time is relatively short while still obtaining high accuracy.

### TECHNICAL FIELD

The invention relates a method of determining activity of a target enzyme of a grain material and a system for determining activity of a target enzyme of a grain material.

### BACKGROUND ART

Enzymes constitute a family of proteins involved in catalyzing chemical reactions within living organisms. As a result of their importance, there are numerous of situations in which it is necessary and/or beneficial to measure enzyme levels, and importantly, enzyme activity.

Within the enzyme family, there are many classes of enzymes that act by facilitating substrate cleavage, for example through hydrolysis or elimination. Such substrate cleavage is usually referred to as substrate degradation and/or digestion.

WO 2016/185533 discloses a method of optical determination of enzyme activity using a biopolymer substrate comprising chemically bonded or entrapped dye. The method may be carried out without allowing a full degradation of the substrate, however this requires that the incubation time used when performing the method should be very accurate according to a preselected incubation time.

Enzymes that degrade or modify polysaccharides are widespread in pro- and eukaryotes and have multiple biological roles and biotechnological applications. Recent advances in genome and secretome sequencing, together with associated bioinformatic tools have enabled large numbers of carbohydrate acting enzymes to be putatively identified. However, there is a paucity of methods for rapidly determination of the biochemical activities of these enzymes.

For many processes, it is desired to know the enzyme activity in seeds and grains, such as cereals, nuts, legumes and spices. Determination of enzyme activity in solid biologic substances is usually performed by extracting the enzyme from the solid biologic substances and determining the enzyme activity in the liquid extract.

The enzyme activity of an enzyme is not directly proportional to the content of the enzyme. Many factors influence the activity of an enzyme. Many of these are known and include for example pH value, temperature, inhibitors and other environmental factors. However, small chemical variations of the enzymes also have large effect and therefore it is necessary to determine the activity of the enzyme and not only the concentration.

However, generally the methods of determination of enzyme activity in seeds are time requiring and/or unreliable.

A method of determining β-Amylase activity in barley or malt is described by E. T. Buttimer et al. in “IMPROVED EXTRACTION AND ASSAY OF B-AMYLASE FROM BARLEY AND MALT” J. Inst. Brena, May-June 1998. Vol. 104. pp. 157-164.

β-Amylase was extracted from barley or malt using four physical techniques to break up grists. The extraction was performed in several steps using a total time of several hours. It was found to be important to ensure that the cells were completely disrupted. The β-Amylase enzyme activity was assayed by measuring the production of reducing sugars from reduced soluble starch, using a PAI-IBAN reagent.

### DISCLOSURE OF INVENTION

An object of the present invention is to provide a method of determining activity of a target enzyme of grain material, such as grain, seed and/or germinated grains, which method is both fast and reliable.

In an embodiment, it is an objective of the invention to provide a method of determining activity of α-Amylase and/or β-Amylase in barley and/or malt, which method is both fast and reliable.

These and other objects have been solved by the inventions or embodiments thereof as defined in the claims and as described herein below.

It has been found that the inventions or embodiments thereof have a number of additional advantages, which will be clear to the skilled person from the following description.

The method of determining activity of a target enzyme of a grain material has surprisingly shown to be both fast and accurate. The method comprises
providing a fluid extract sample of the grain material using a preselected extraction procedure,
providing a dyed and/or chromogenic substrate for the target enzyme,
subjecting the fluid extract sample to the substrate for a preselected incubation time, and
determining the target enzyme activity of the grain material e.g. using UV/Vis spectrophotometry.

By ensuring that the extraction procedure is a preselected procedure, it has been found that the extraction need not be complete or even near complete. If the extraction procedure is performed according to a preselected procedure, it may be sufficient to extract a fraction of the target enzyme and still determine the total enzyme activity in the grain material. Thereby the determination may be performed very fast and at the same time with a desirable high accuracy.

The preselected extraction procedure may comprise the following procedural steps
milling the grain material to a predetermined particle size distribution,
preparing an extract from the milled substance by a method comprising thoroughly mixing a preselected amount of the milled grain material with a preselected amount of liquid extraction buffer and allowing the enzyme extraction for a preselected extraction time of up to about 20 minutes, and
taking the fluid extract sample of the liquid extraction buffer.

The step of determining the enzyme activity of the grain material may comprise following procedural steps
determining an absorbance parameter of dye released from the substrate into the fluid extract sample and/or colored reaction product formed,
correlating the determined absorbance parameter to a standard curve associated to the preselected extraction procedure and representing absorbance parameter as a function of target enzyme activity.

In addition to providing a fast and reliable determination of activity of a target enzyme of a grain material, the method has been found to be very simple to carry out and does not require special knowledge. The method may for example be carried out by an ordinary staff member instructed to follow a manual specifying the various steps of the method.

It should be emphasized that the term “comprises/comprising” when used herein is to be interpreted as an open term, i.e. it should be taken to specify the presence of specifically stated feature(s), such as element(s), unit(s), integer(s), step(s) component(s) and combination(s) thereof, but does not preclude the presence or addition of one or more other stated features.

Reference made to “some embodiments” or “an embodiment” means that a particular feature, structure, or characteristic described in connection with such embodiment(s) is included in at least one embodiment of the subject matter disclosed. Thus, the appearance of the phrases “in some embodiments” or “in an embodiment” in various places throughout the specification is not necessarily referring to the same embodiment(s). Further, the skilled person will understand that particular features, structures, or characteristics may be combined in any suitable manner within the scope of the invention as defined by the claims.

The term “substantially” should herein be taken to mean that ordinary product variances and tolerances are comprised.

Throughout the description or claims, the singular encompasses the plural unless otherwise specified or required by the context.

All features of the invention and embodiments of the invention as described herein, including ranges and preferred ranges, may be combined in various ways within the scope of the invention, unless there are specific reasons not to combine such features.

The term “material” means any material obtainable from one or more grains including grains, seeds, germinated grains, fractions thereof and/or parts thereof.

The term “incubation” means to provide an enzymes sample in contact with a substrate to allow enzyme activity, such a degradation/digestion of optional enzyme of the sample.

The term “incubation time” means the time of incubation. I.e. the time of allowing the sample in contact with the substrate until the sample is taken away from the substrate sample or the enzyme activity is stopped by other means e.g. by adding a stop agent, such as sodium carbonate solution.

A stop agent is an agent that is terminating the enzyme activity of the enzyme e.g. by changing pH level.

The grain material may be any type of vegetable grain material or fractions thereof. Examples of grains or seeds include cereals, nuts, legumes, spices and any combinations comprising at least one of these.

Legumes advantageously include one or more seeds/grains of alfalfa, clover, beans, peas, chickpeas, lentils, lupins, mesquite, carob, soybeans, peanuts, and tamarind.

In an embodiment, the target enzyme is α-Amylase, β-Amylase, Limit Dextrinase, Beta-glucanase, lipase, xylanase, cellulase, protease or any combinations comprising one or more of these.

The invention is particularly advantageous, where the grain material are substantially dry, such as with a water content of about 15% by weight or less, such as of about 10% by weight or less, such as of about 5% by weight or less.

Usually grain material are kept free of moisture and/or are dried to a water content for storing which is less than 5% by weight, or even lower water content such at about 4% by weight or is or about 3% by weight or less. The enzymes in such low water containing and optionally stored grain material, tends to be more difficult to extract from the grain material. However, using the present invention the method of determining activity of a target enzyme of the low moisture grain material may be performed surprisingly fast and with a desired accuracy.

In an embodiment, the method comprises determining activity of a target enzyme of a grain material with a water content less than about 5% by weight.

The invention has found to be especially useful where the grain material are selected from barley, wheat, rye or oat, sorghum, germinated versions thereof e.g. malt or any combinations comprising one or more of these.

To ensure a representative grain material, it is advantageous to select a number of seeds, grains and/or germinated grains, e.g. a spoon full or a weight amount thereof and subject these seeds/grains/germinated grains to the milling process.

The selected particle size is advantageously determined by sieving and/or laser diffraction.

Advantageously, the step of milling the grain material comprises milling the grain material to a particle size where at least 90% by weight, such as at least 9 5% of the grain material passes a sieve of 0.5 mm (mesh 35) or less, such as, a sieve of 0.4 mm (mesh 40) or less, such as, a sieve of 0.3 mm (mesh 50) or less, such as, a sieve of 0.2 mm (mesh 70) or less.

To ensure a desired particle distribution, it is advantageous to use a mill adapted for reaching the desired particle size distribution. Examples of suitable mills include a disc mill, a grist mill, a coffee mill and/or a Hammer mill.

For example when using a disc mill the distance between the discs may advantageously be set to the desired maximal particle size.

In an embodiment, the milling is performed using a Bühler Miag disc mill, DLFU (Bühler GmbH, Germany). The gap between the grinding discs may advantageously be 0.1-0.5 mm, such as about 0.2 mm.

Advantageously the milling comprises milling a preselected amount of grain material for a preselected time.

It is preferred that the mill type used and the preselected milling time is substantially identical to the mill and milling time used for preparation of the standard curve as described further below.

If the grain material is adapted for being used in a milled condition, it may be advantageous to use the same mill and milling condition in the assay for determining the target enzyme activity.

After milling, a preselected amount of the milled grain material may be taken from the milled grain material. This may for example be done by weight or volume, e.g. using a spoon with a preselected volume. Using a spoon may be simple and fast and may in an embodiment be desired. The preselected amount of the milled grain material may advantageously be at least about 10 mg, such as from about 0.1 g to about 5 g, such as from about 0.3 g to about 1 g of the milled grain material.

The taken amount of milled grain material may then be thoroughly mixed with the preselected amount of liquid extraction buffer e.g. by shaking and/or stirring. The preselected amount of extraction buffer may advantageously be from about 5 ml to about 0.5 L per gram milled grain material, such as from about 10 ml to about 0.2 L per gram milled grain material.

In an embodiment, the amount of preselected amount of milled grain material is from 300 to 1000 mg and the amount of liquid extraction buffer is 10-50 ml.

The liquid extraction buffer is advantageously selected in dependence of the target enzyme, to ensure that the target enzyme activity is determined at a desired pH value. The target enzyme activity may depend on the pH value. It is well known that for many enzymes, its activity are affected by changes in pH value. The most favorable pH value—the point where the enzyme is most active—is known as the optimum pH.

The optimum pH value may vary greatly from one enzyme to another.

The optimal pH values for a number of potential target enzymes are listed in FIG. 1.

Advantageously the aqueous buffer having a pH value of from about 4 to about 10, such as from about 5 to about 8.

For grain material enzymes, the preferred pH value of the liquid extraction buffer is about 5.5-6.5. For most enzyme, it is desired that the pH value is not below 4.5 since a very low pH value may inactivate the enzyme.

To increase the extraction effectivity, it may be desired that the liquid extraction buffer comprises a reducing agent, which may improve the effect of breaking bonds in the grain material to release the target enzyme.

The reducing agent may advantageously comprise Dithiothreitol (DTT) and/or tris(2-carboxyethyl)phosphine (TCEP).

Especially DTT has been found to be effective as a reducing agent to break disulfide bonds within and between proteins, which may thereby result in release of target enzyme.

In an embodiment, the liquid extraction buffer comprises cysteine, which has shown to participate in releasing target enzyme.

The thoroughly mixing of a preselected amount of the milled grain material with a preselected amount of liquid extraction buffer advantageously comprises, shaking or stirring the mixture for a part of or for the entire extraction time. For most effective and fast release the preselected amount of the milled grain material and the preselected amount of liquid extraction buffer is advantageously shaken in a shaker for at least about 50%, such as at least about 80%, such as at least about 90% of the extraction time.

The shaking may e.g. be performed in an overhead shaker e.g. with 20 rpm or more.

In an embodiment, the preselected extraction time may be even less than 20 minutes, which thereby result in an even faster determination of target enzyme activity. It has been found that even when the extraction time is between 1 and 15 minutes the target enzyme activity may be determined with a high accuracy.

In an embodiment, the preselected extraction time is up to about 15 minutes, such as up to about 10 minutes, such as up to about 8 minutes, such as from about 2 to about 6 minutes.

Due to the present invention, it has been found to be sufficient to extract a fraction of the target enzyme from the milled grain material. Such as a fraction of 1-50% by activity, such as 5 to 25% by activity, such as about 10% or less by activity.

Due to this realization, the extraction may be performed very fast and in addition, it has been found that heating of the liquid extraction buffer may be required. In an embodiment, the extraction is performed at a temperature from about 10 to about 40° C., such as from about 15 to about 30° C., such as from about 18 to about 24° C.

Typically, the extraction may be performed at room temperature (21° C.), which add to keep the extraction simple and fast.

After the extraction, the preselected extraction time may advantageously be terminated by taking the fluid extract sample of the liquid extraction buffer. It is desired that the fluid extract sample taken of the liquid extraction buffer is essentially free of particulate grain material to ensure that no further target enzyme is extracted.

The taken (withdrawn) fluid extract sample of the liquid extraction buffer may be a portion of the liquid extraction buffer or substantially the entire amount of liquid extraction buffer—i.e. the amount that may be filtered free of the remaining solid parts of the grains.

The fluid extract sample may be taken of the liquid extraction buffer using any method. This may for example be performed by allowing the container with the liquid extraction buffer with the milled grain material stand still for precipitating particulate grain material and thereafter withdrawing the fluid extract sample from the top of the container. Alternatively or in addition, the fluid extract sample may be filtered e.g. using a filter syringe and/or by centrifuging.

Advantageously, the fluid extract sample is added to the substrate within 10 minutes from the fluid extract sample has been taken from the liquid extraction buffer, preferably within 5, such as within 2 minutes, such as immediately after withdrawing the fluid extract sample from the liquid extraction buffer. Thereby any risk of undesired reduction of enzyme activity, e.g. due to denaturation or other damage of the enzyme may be reduced.

During the extraction, several different components may be released into the same extraction buffer where they may interact with each other. Modes of such interactions, especially with regard to enzymatic activity, may include degradation, inhibition and/or synergistic or antagonistic effects which may influence the enzyme activity of the target enzyme. This may be accounted for by using identical extraction buffer and condition when determining the standard curve.

The desired incubation time depends largely of the substrate used.

The substrate for performing the enzyme activity assay is preferably identical to the substrate used for generating the standard curve as further described below. In the same way, it is desired that also the amount of substrate relative to the fluid extract sample is identical to the amount of substrate relative to the reference fluid extract sample as used in the generation of the standard curve.

A desired incubation time may depend on the target enzyme. For example for alpha-amylase of wheat flour, a desired incubating may be up to about 45 minutes.

The incubation time may be preselected or it may be within a preselected range.

Advantageously, the preselected incubation time is not increasing about 50 minutes to thereby keep the determination of target enzyme activity relatively fast. Preferably, the preselected incubation or the preselected range of incubation time is advantageous about 30 minutes or less, such as about 20 minutes or less. It has been found that the preselected incubation time optimally is in the range from about 1 minute to about 20 minutes.

It has been found that where the target enzyme is beta amylase a desired preselected incubation time is from about 5 to about 10 minutes, such as about 7 minutes. Where the target enzyme is alpha amylase the preselected incubation time may be less than 10 minutes and even as low as 5 minutes or even less.

Dyed and/or chromogenic substrates are well known in the art and the skilled person will be capable of selecting a suitable substrate.

The dyed and/or chromogenic substrate is advantageously specific for the target enzyme. A specific substrate should in this context be interpreted to be a substrate that is exclusively digested by the target enzyme and not digestible by other enzymes present in the fluid extract sample.

The selected dyed and/or chromogenic substrate may be a gelled biopolymer substrate comprising cross-linked polymeric biomolecules selected from polynucleotides, polypeptides, polysaccharides or any combinations thereof, preferably the dyed and/or chromogenic substrate is an aerogel or a xerogel.

In an embodiment, the substrate is a substrate as described in WO2016/188533.

Where a dyed substrate is used, it is desired that the method comprises filtering of solid parts of the substrate after digestion and prior to determining the absorbance parameter of dye or dye bound to small degraded substrate fragments released from the substrate into the fluid extract sample.

The dye released to the fluid extract sample may be in the form of non-bonded dye incased in a crosslinked polymer or preferably in the form of dye bound to short fragments of the substrate, which have been digested by the target enzyme.

It has been found to be very effective to terminate the incubation time by filtering of the solid, non-dissolved parts of the substrate, to thereby ensure that no further digestion takes place such that no further release of dye occur. The digestion is terminated immediately and thereby the incubation time may be effectively controlled.

Where a chromogenic substrate is used the target enzyme provides that a colored reaction product is formed from the chromogenic substrate.

The chromogenic substrate may be a dissolvable chromogenic substrate, such as a colorless chromogenic substrate. Where a chromogenic substrate is used, it is generally desired to omit filtering, since this otherwise may be a superfluous step.

In an embodiment, the incubation time may be terminated by adding a stop agent.

The enzyme activity of the target enzyme is advantageously determined in enzyme units. Such enzyme units are relative units e.g. defined by the standard curve. The standard curve may e.g. define the enzyme unit as enzyme unit per ml fluid extract sample and/or as enzyme unit per mg grain material.

The enzyme units may include standard enzyme assay units.

The enzyme unit may in an embodiment be defined by the user.

The enzyme unit may be an enzyme assay unit specifically linked to a particular enzyme, such as “DU” for α-amylase. In an embodiment the enzyme unit is parts-per million (ppm).

The determining of the target enzyme activity of the grain material advantageously comprises measuring the absorbance parameter of the fluid extract sample with released dye or formed colored reaction product, wherein the absorbance parameter comprises at least one wavelength absorbable by the dye.

In an embodiment, the standard curve further is associated to the selected dyed and/or chromogenic substrate and preselected incubation time.

In an embodiment, the method further comprises generating the standard curve. The generation of the standard curve advantageously comprises,
providing a plurality of grain material reference samples with different and known enzyme activity of the target enzyme,
providing a fluid extract reference sample of each of the respective grain material reference samples using the preselected extraction procedure,
providing a selected dyed and/or chromogenic reference substrate for the target enzyme for each of the fluid extract reference sample,
subjecting each of the respective fluid extract reference samples to the respective selected dyed and/or chromogenic reference substrates for a preselected incubation time,
determining an absorbance parameter of dye released from and/or colored reaction product formed in each of the respective reference substrates into the fluid extract sample,
performing a regression including points of respective pairs of determined absorbance parameter and corresponding, known enzyme activity to provide a linear standard curve.

The grain material reference samples with different and known enzyme activity of the target enzyme are advantageously grain materials of same type of grain materials to be analyzed by the method. For example if the method comprises determining a target enzyme in wheat material, the reference samples are advantageously wheat material reference samples with different and known enzyme activity of the target enzyme. Where the method comprises determining a target enzyme in barley material, the reference samples are advantageously barley material reference samples with different and known enzyme activity of the target enzyme and so on.

The known target enzyme activity in the reference grain material may be given in any kind of enzyme activity units e.g. as discussed above. In an embodiment, the known enzyme activity of the reference grain materials are given in standardized units.

Reference grain material samples may for example be obtained by performing a full extraction of selected samples and analyzing these for enzyme activity and/or reference grain material samples may be purchased from various supplier, such as MAPS (The Malt Analytes Proficiency Testing Scheme) and IFBM (French Institute of Brewing and Malting).

Advantageously, the plurality of grain material reference samples with different and known enzyme activity of the target enzyme comprises at least 3 grain material reference samples, such as at least 5 grain material reference samples, such as at least 8 grain material reference samples. It has been found that, in most situations 5-10 reference samples are sufficient to provide a very accurate standard curve.

In addition, it has been found that the standard curve is substantially or practically linear, which make the required number of reference sample very low.

In an embodiment, the regression is a linear regression. Alternatively a nonlinear regression may be applied.

Advantageously the selected dyed and/or chromogenic reference substrate is identical to the selected dyed and/or chromogenic substrate.

The preselected reference incubation time for the fluid extract reference samples is/are advantageously identical to the incubation time for the fluid extract sample(s). Thereby, the highest accuracy is obtained. However, it has been found that small variations may be acceptable. In an embodiment, two or more standard curves obtained using different incubation time may be provided. Thereby it may be possibly to interpolate or even extrapolate based on these standard curves to an actual incubation time used for the fluid extract sample(s).

In an embodiment, the preselected incubation time for the fluid extract reference samples is/are from 5 minutes shorter to 5 minutes longer than the incubation time for the fluid extract sample, such as from 1 minute shorter to 1 minute longer, such as from 0.5 minutes shorter to 0.5 minutes longer.

The preselected reference extraction procedure for each of the respective grain material reference sample may advantageously be as the preselected extraction procedure for the grain material examined for target enzyme activity.

In an embodiment, two or more standard curves are obtained using different preselected reference extraction procedures for the respective grain material reference samples, which preselected reference extraction procedures differs in extraction time only. Thereby it may be possibly to interpolate or even extrapolate based on these standard curves to an actual extraction time of the extraction procedure for the grain material examined for target enzyme activity.

This provided a high freedom for the user, while simultaneously ensuring a high accuracy.

The absorbance parameter may be any kind of absorbance parameter, such as, a measure of reflected (not-absorbed) light, a measure of transmitted (not-absorbed) light, a measure of total intensity, a measure of intensity of one or more wavelengths, a measure of intensity profile (intensity as a function of wavelengths) and/or a combination comprising at least one of these.

The absorbance parameter may be determined by a spectroscope comprising a light source and an optical reader.

The light source used for performing the determination of the absorbance parameter, may be any light source comprising at least one wavelength absorbable by the dye. Advantageously the light source comprised a xenon light source, a xenon-mercury light source and/or a diode. A xenon light source or a xenon-mercury light source may be especially preferred where a broad wavelength range is desired e.g. covering UV to IR, such as in the range of 185-2000 nm.

The reader for reading the absorbance parameter may be any optical reader capable of reading at least a fraction of not absorbed light, preferably comprising at least one wavelength absorbable by the dye.

The regression comprises at least an interpolation between two or more pairs of determined absorbance parameter and corresponding, known enzyme activity. In an embodiment, the regression comprises an extrapolation from one or more points of respective pairs of determined absorbance parameter and corresponding, known enzyme activity.

### BRIEF DESCRIPTION OF THE EXAMPLES AND DRAWING

The invention is being illustrated further below in connection with a few examples and embodiment and with reference to the drawings in which:

FIG. 1 shows a table of pH optimum for a number of enzymes.

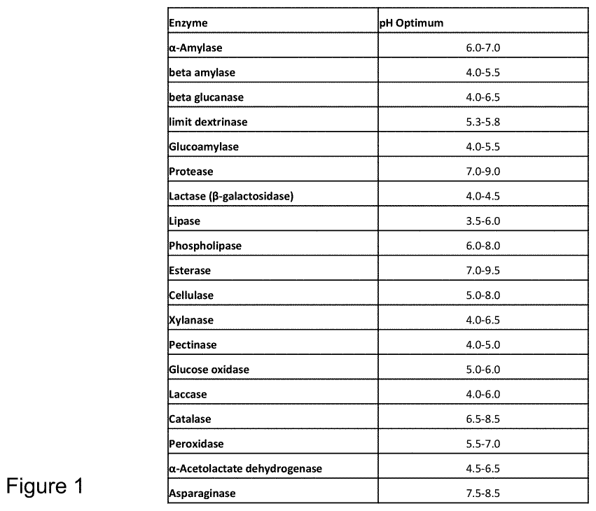

FIG. 2a illustrates a system for determining activity of a target enzyme of a sample.

FIG. 2b illustrates a system for determining activity of a target enzyme of a grain material.

FIG. 3a illustrates a standard curve associated to a preselected extraction and digestion procedure.

FIG. 3b illustrates a further standard curve associated to a preselected extraction and digestion procedure.

FIG. 4a illustrates data points for five standard curves, each associated to respective preselected extraction times for extracting target enzyme from a grain material.

FIG. 4b illustrates five standard curves for five different selected extracting times.

FIG. 5a illustrates data points for six standard curves, each associated to respective preselected reference incubating times for enzymatic actions involving a substrate by respective samples containing known amount of target enzyme.

FIG. 5b show standard curves of concentration as a function of absorbance for a number of incubation times.

FIG. 6 shows determined absorbance parameter values as a function of the Ring trial WK values obtained in example 2 an example 3.

FIG. 7 shows determined absorbance parameter values as a function of the Ring trial WK values obtained in example 4.

FIG. 8 shows determined absorbance parameter values as a function of the Ring trial WK values obtained in example 5.

FIG. 9 shows determined absorbance parameter values as a function of the Ring trial WK values obtained in example 6.

FIG. 10 shows determined absorbance parameter values as a function of the DU values obtained in example 7.

FIGS. 11a and 11b shows determined absorbance parameter values as a function of the BU3/ml and BU3/g values obtained in example 8.

FIGS. 12a and 12b shows determined absorbance parameter values as a function of the BU3/ml and BU3/g values obtained in example 9.

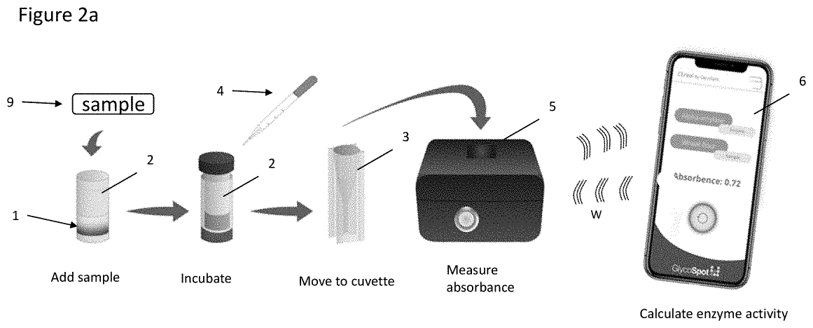

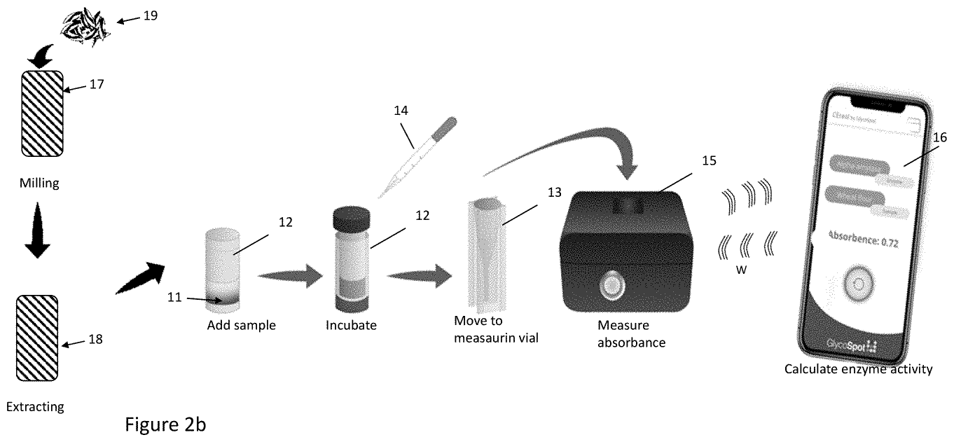

The system illustrated in FIG. 2a for determining activity of a target enzyme of a sample, comprises a container 2 containing a substrate 1, a cuvette 3, a pipette 4, a reader 5 and a tablet 6, such as a smartphone.

In use, a sample 9 comprising a target enzyme is added into the container 2 comprising the substrate for the target enzyme. The sample is advantageously a liquid sample, e.g. obtained from a biological matter, e.g. by extraction, dissolving or diluting or without any pretreatment. The container 2 act as an incubator and the sample is incubated with the substrate for an incubating time, also referred to as an actual incubation time or the incubation time associated to this sample. The incubation time may be terminated by adding a stop agent to the container 2. After incubation, a portion of the sample is taken out from the container 2 using the pipette 4 and added to the cuvette 3. The cuvette 3 is inserted into the reader 5, which in this example is an optical reader configured for reading absorbance. The reader 5 is reading and/or determining an absorbance parameter value associated to enzymatic actions of the target enzyme involving the substrate. The absorbance value is transmitted e.g. wireless as indicated with the waves W to the tablet 6. The tablet may also be configured to transmit data to the reader 5, such as data representing the actual incubation time. The reader 5 may be calibrated e.g. using a blank sample.

A computer of the reader 5 and a computer of the tablet 6 form parts of the computer system and they may be as described above. The computer system comprises a memory storing reference data representing one or more standard curves, such as at least two sets of reference data, each set of reference data comprises data representing a standard curve for said parameter associated to said enzymatic actions of said target enzyme involving said substrate as a function of enzyme activity and correlated to an incubation time and an incubation time attribute representing said incubation time, wherein said at least two sets of reference data having different incubation time attribute representing different incubation time

The system illustrated in FIG. 2b for determining activity of a target enzyme of a grain material, comprises a milling device 17, an extracting device 18, a container 12 containing a substrate 11, a cuvette 13, a pipette 14, a reader 15 and a tablet 16, such as a smartphone.

In use, a sample of grain material 19 comprising a target enzyme is milled in the milling device 17 e.g. as described above. The milled material or at least a portion of the milled material is moved to the extracting device 18 for extracting target enzyme into an extraction liquid e.g. as described above. At least a portion of the extraction liquid is moved to the container 12 e.g. using a method as described. The container 12 act as an incubator and the extracting liquid with extracted target enzyme is incubated with the substrate for an incubating time, also referred to as an actual incubation time or the incubation time associated to this sample. The incubation time may be terminated by adding a stop agent to the container 12. After incubation, a portion of the liquid is taken out from the container 2 using the pipette 4 and added to the cuvette 13. The cuvette 13 is inserted into the reader 15, which in this example is an optical reader configured for reading absorbance. The reader 15 is reading and/or determining an absorbance parameter value associated to enzymatic digestion of the substrate by the target enzyme. The absorbance value is transmitted e.g. wireless as indicated with the waves W to the tablet 16. The tablet may also be configured to transmit data to the reader 15, such as data representing the actual incubation time. The reader may be calibrated e.g. using a blank sample.

A computer of the reader 15 and a computer of the tablet 16 form parts of the computer system and they may be as described above. The reader 15 and the tablet 16 may be as described for the system of FIG. 2a.

FIG. 2 illustrates a system for determining activity of a target enzyme of a grain material.

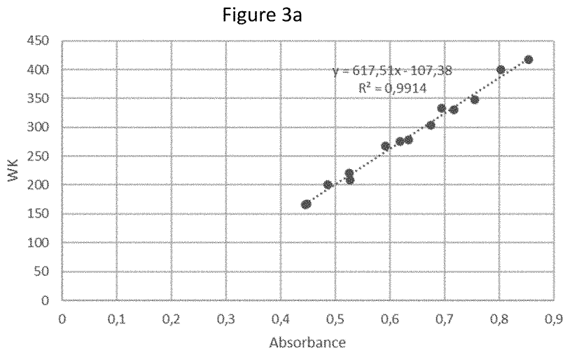

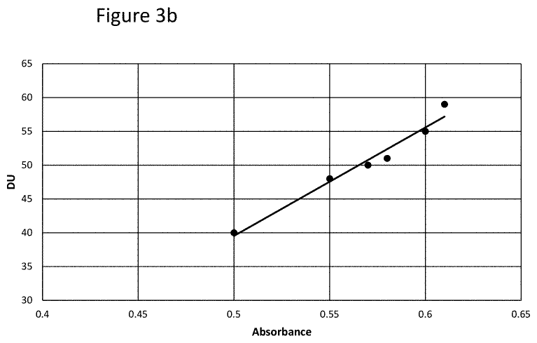

The standard curve associated to a preselected extraction and digestion procedure illustrated in FIG. 3a represents beta-amylase enzyme activity in terms of diastatic power (unit Windisch-Kolbach (WK)) as a function of absorbance parameter at a preselected incubation time. The standard curve may be obtained by determining the absorbance parameter value for a number of samples with known beta-amylase enzyme activity (WK), using the preselected incubation time. Where the beta-amylase enzyme activity is determined in grain, the grain may be extracted as described above using a preselected extraction time.

The standard curve associated to a preselected extraction and digestion procedure illustrated in FIG. 3b and represent alpha amylase enzyme activity in terms of dextrinization units (DU) as a function of absorbance parameter at a preselected incubation time. The standard curve may be obtained by determining the absorbance parameter value for a number of samples with known alpha amylase enzyme activity (DU), using the preselected incubation time. Where the alpha amylase enzyme activity is determined in grain, the grain may be extracted as described above using a preselected extraction time.

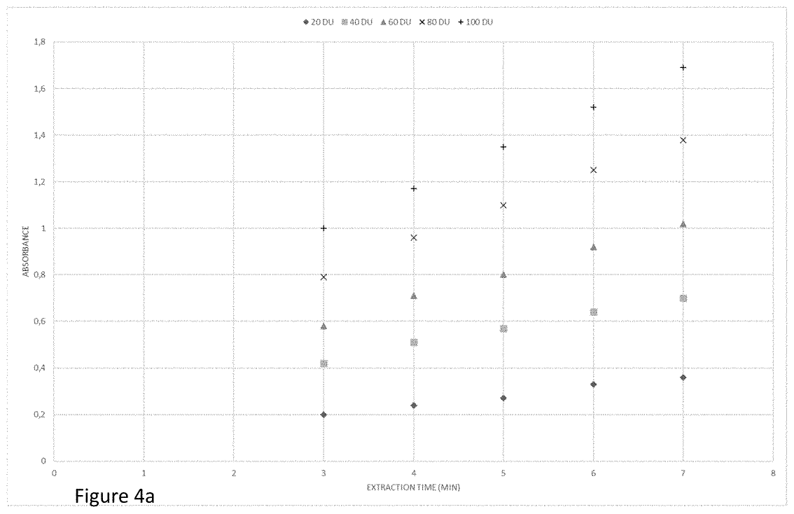

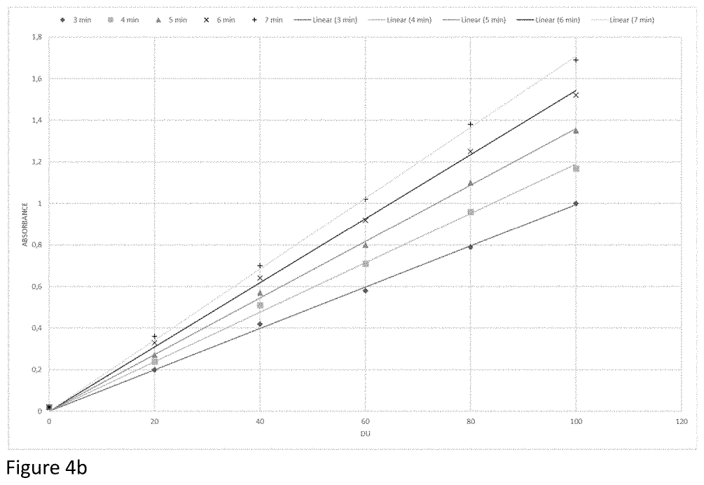

The five standard curves illustrated by the data points in FIG. 4a, show standard curves for alpha amylase enzyme activity in terms of dextrinization units (DU) as a function of absorbance parameter where the standard curves are associated to respective preselected extraction times for extracting target enzyme, here alpha amylase, from a grain material.

The curves may be obtained by determining absorbance parameter values for a number of grain material samples with different and known alpha amylase enzyme activity (DU) and at different extraction times. Preferably, the incubation time used for the determinations are identical. In this example the absorbance parameter values for five grain material samples with different and known alpha amylase enzyme activity (DU)—here 20, 40, 60, 80 and 100 DU respectively were determined at extraction times of 3, 4, 5, 6 and 7 minutes.

It will be observed that the data points for each standard curve are lying on a substantially straight line.

The five standard curves illustrated in FIG. 4b for five different selected extraction times are provided using the data points of FIG. 4a. The five standard curves are each associated to respective preselected reference extracting times for extracting of alpha amylase and show absorbance parameter as a function of extraction time of extracting of alpha amylase from grain.

It can be seen that the standard curves are practically linear, which makes it relatively simple to determining a best fit standard curve for any extraction time, such as an extraction time associated to a sample under examination, where the extraction time differs from the extraction times used in generating the standard curves—e.g. an extraction time between extraction times used in generating the standard curves.

After having determined the best fit standard curve for such sample under examination, the target enzyme activity may be determined from the absorbance parameter value determined for the sample under examination.

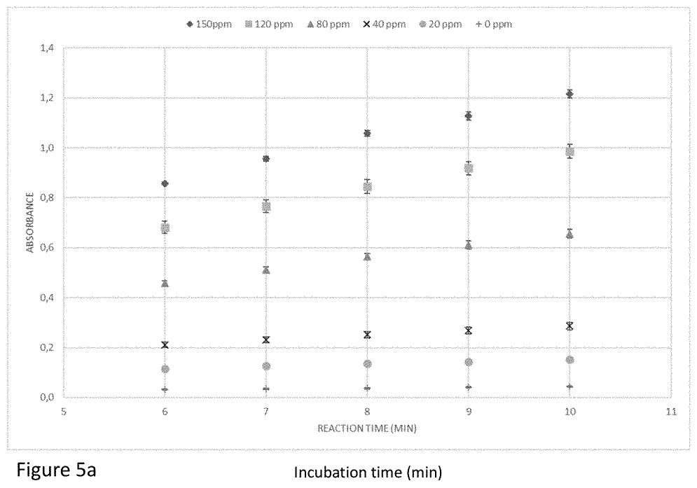

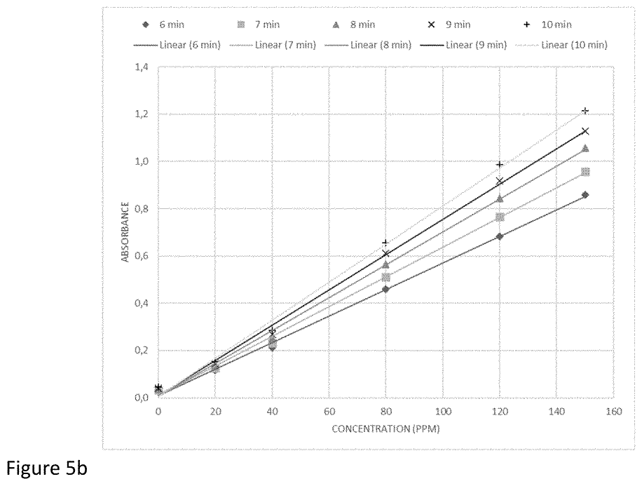

The six standard curves illustrated by data points in FIG. 5a, is each associated to respective preselected reference incubating times for enzymatic actions involving a substrate by respective samples containing known amount in ppm of target enzyme.

The curves may be obtained by determining absorbance parameter values for a number of samples with different and known amounts of target enzyme (ppm) and at incubation times. It will be observed that the data points for each standard curve are lying on a substantially straight line.

The standard curves illustrated in FIG. 5b of concentration as a function of absorbance for a number of incubation times are obtained using the data points of FIG. 5a.

It can be seen that the six standard curves are practically linear, which makes it relatively simple to determining a best fit standard curve for any incubation time, such as an incubation time associated to a sample under examination, where the incubation time differs from the incubation times used in generating the standard curves—e.g. an incubation time between incubation times used in generating the standard curves.

After having determined the best fit standard curve for such sample under examination, the target enzyme activity may be determined from the absorbance parameter value determined for the sample under examination.

FIG. 5a. illustrates six standard curves, each associated to respective preselected reference incubating times for digestion a substrate by a fluid extract sample and representing absorbance parameter as a function of incubation time for samples with known target enzyme activity, wherein the respective preselected incubating times differs from each other with respect to preselected reference incubation time.

FIG. 5b show the concentration as a function of absorbance for a number of incubation times.

In the following examples the terms “Distatic power” and “Ring trial” has the following meaning:

Diastatic power: The malts ability to break down starches into simpler fermentable sugars during the mashing process. It is measured in Windisch-Kolbach (WK), IoB or Lintner. By this definition, this is the join action of all amylases, mostly alpha amylase and beta amylase, which are the main sugar producing enzymes during mashing. However, since alpha amylase is always in excess, diastatic power is somehow correlated with beta amylase.

Ring trial: Validation of the methods of one's lab through the collaboration of typically 10-100 different labs. It includes methods for diastatic power and alpha amylase. The main ring trials in Europe are organized by the Institute Francais de Boissons, de la Brasserie et de la Malterie (IFBM), LGC Standards, known as the Malt analytes scheme (MAPS), and the ones organized by the VLB institute in Berlin.

### Example 1

Constructing a Calibration Curve for Determining Alpha-Amylase Activity in Barley Malt Samples

The construction of calibration curves is based on grain reference samples with known reference values of enzyme activities supplied by recognized certification bodies such as EBC and IFBM.

13 samples barley grain reference samples with known alpha-amylase activity is obtained from the European Brewery Convention and the French Institute of Beverages, Brewing and Malting. The sample is as follows:

| Barley reference | known alpha-amylase | known beta-amylase |
|---|---|---|
| sample # | activity (DU) | activity (WK) |
| 1 | 59 | 258 |
| 2 | 55 | 595 |
| 3 | 51 | 250 |
| 4 | 48 | 222 |
| 5 | 50 | 390 |
| 6 | 40 | 203 |
| 7 | 55 | 267 |
| 8 | 64 | 271 |
| 9 | 64 | 269 |
| 10 | 66 | 334 |
| 11 | 64 | 331 |
| 12 | 24 | 223 |
| 13 | 49 | 269 |

The dextrinizing units (DU) is a standard unit in the malt industry and specified in EBC Method “4.13 α-AMYLASE CONTENT OF MALT (IM)—2006”. The diastatic power measured in Windisch-Kolbach units is specified in EBC method 4.12.1. DIASTATIC POWER OF MALT BY SPECTROPHOTOMETRY (MANUAL METHOD)—2018.

A fluid extract reference sample is obtained from each barley malt reference sample using a preselected extraction procedure.

The preselected extraction procedure is as follows:
The samples are milled by use of a lab mill, e.g. Bühler Miag Disc Mill
300 mg of the milled grain material from each barley reference sample is mixed with 50 ml liquid extraction buffer. Each sample of liquid extraction with barley reference sample is arranged in a shaker. Let the enzyme extraction go on for 5 minutes with gentle shaking at 20 rpm using an overhead shaker, for allowing the enzyme extraction for 5 minutes.

The composition of the liquid extraction buffer was prepared according to the following scheme:

Dissolve 134.1 g of malic acid, 70 g of NaOH, and 58.4 g of NaCl in 900 mL of water (deionized or ultra-pure). Next, add 6.0 g CaCl₂)*2H₂O until complete dissolution. The pH should be adjusted to pH 5.4 by dropwise addition of concentrated (4 mol L⁻¹) NaOH or HCl. Sodium azide (1.0 g) can be added as a preservative (antimicrobial agent). If sodium azide is added, the reagent will be stable for more than one year. If not, the shelf-life of the reagent will be 2 weeks if preserved at 4° C. This concentrated buffer should be diluted 200 times (e.g. 50 mL of concentrated solution for a final volume of 1000 mL) in order to be used in the extraction protocol.

The extraction was performed at room temperature (21° C.).

Thereafter a fluid extract reference sample of 250 microliter is taken of each liquid extraction buffer with extracted enzyme using a pipette suitable for the purpose. Each sample was carefully aspirated from the liquid phase and thereby minimizing the risk of introducing solid matter in the pipette tip. After proper aspiration, the sample was dispensed in the provided filter vial containing the substrate and further diluted by adding 250 microliter of the above mentioned buffer.

The substrates were produced by first dyeing polysaccharides with one of the four chlorotriazine dyes (red, blue, green or yellow) via nucleophilic aromatic substitution. The polysaccharides were then cross-linked with 1,4-butanediol diglycidyl ether via base-catalysed epoxide opening. The resulting materials are hydrogels, which can be easily dispensed using syringes into 96-well filter plates or vials. Additional info about production of such substrate may be found in Kračun, S. K., et al., A new generation of versatile chromogenic substrates for high-throughput analysis of biomass-degrading enzymes. Biotechnology for Biofuels, 2015. 8(1): p. 70.

The alpha-amylase in the sample will digest the substrate and develop a blue colored solution with an intensity related to the enzymatic activity as a function of incubation time.

The incubation was performed at room temperature applying a reference incubating time of 5 minutes with gentle shaking at 20 rpm using an overhead shaker.

For each sample, the reference incubation time is terminated by suction of the fluid extract reference sample with released substrate fragments and dye over the filter in each container and collecting the fluid extract reference sample in a cuvette.

A further cuvette is supplied by a “blind” sample of the liquid extraction buffer without any extracted enzyme. Instead of a blind sample of liquid extraction buffer, a blind sample of pure water could have been used.

Measurement

Each reference sample including the blind is subjected to a spectrometer to determine the intensity of transmitted light of a light source comprising a wavelength absorbable by the dye.

An absorbance parameter is determined for each reference samples by withdrawing the light intensity transmitted through the blind from the light intensity transmitted through the respective samples.

Thereafter the respective absorbance parameter is plotted as a function of alpha amylase activity (DU).

| Barley | known alpha- | absorbance (# transmitted |
|---|---|---|
| reference | amylase | light intensity − blind transmitted |
| sample # | activity (DU) | light intensity) |
| 1 | 59 | 1.22 |
| 2 | 55 | 1.2 |
| 3 | 51 | 1.16 |
| 4 | 48 | 1.1 |
| 5 | 50 | 1.14 |
| 6 | 40 | 1.0 |

The absorbance is determined according to the formula:

$$A = {log}_{10} \frac{{Φ}_{e}^{i}}{{Φ}_{e}^{t}} = - {log}_{10} T ,$$

where
Φₑ^{t} is the radiant flux transmitted by that material;
Φₑ^{j} is the radiant flax received by that material,
T=Φₑ^{t}/Φₑⁱ is the transmittance of that material.

### Example 2

Ring Trial Validation—Extraction Time

Ring trial validations using five different milled malt samples having different WK values were performed.

The five samples of milled barley malt with different and known WK value were as follows:

| Sample | Sample 1 | Sample 2 | Sample 3 | Sample 4 | Sample 5 |
|---|---|---|---|---|---|
| WK | 203 | 267 | 334 | 379 | 60 |

The samples were tested according to the following protocol
Weigh 200 mg of milled malt.
Extraction with extraction buffer for 10 minutes at 35° C.
Syringe filtration with 0.45 μm filter. Collect the filtrate.
Incubation of 250 μL filtrate with substrate for an incubation time at 35° C.
Stop the enzymatic cleaving by adding a stopping reagent
Read the resulting liquid at 410 nm in a spectrophotometer.

The extraction buffer used was the maleic acid based extraction buffer described in example 1. For each sample, 25 mL of extraction buffer was used together with 35 mg Dithiothreitol.

The substrate used was a glucose-based oligosaccharide conjugated with a chromophore, able to absorb visible light at 410 nm when the pH is higher than 9, mixed with a glucosidase. By the action of the enzyme of interest, in this case beta amylase, the oligosaccharide is then accessible to the glucosidase, which then makes the chromophore free of glucose molecules. Such chromophore would then change color by the addition of the stopping reagent, and its intensity will depend directly on how much the enzyme of interest cleaves the oligosaccharide.

The incubation time used was 7 minutes.

The stopping agent used was an alkaline sodium carbonate solution.

The spectrophotometer used was the spectrometer sold by Glycospot under the trade name SIRIUS™.

The absorbance parameter for each sample was determined by withdrawing the light intensity transmitted through a blind from the light intensity transmitted through the respective samples.

Each test was performed in triplicate and the average absorbance parameter values were determined.

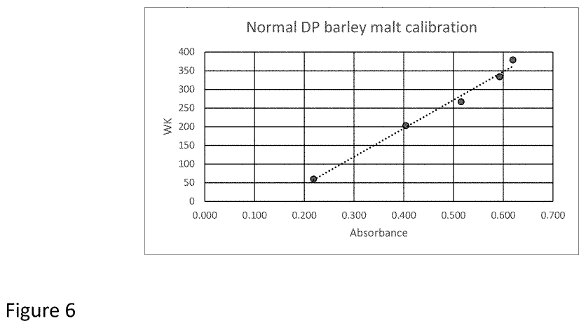

Thereafter the respective absorbance parameter values were plotted as a function of the Ring trial WK values. The plot is shown in FIG. 6.

The result indicates a good correlation with the ring trial samples even with a relatively low extraction time, here 10 minutes. It is assessed that an even lower extraction time could have been applied and still maintaining a good correlation with the ring trial.

### Example 3

Reference Curve for Barley Malt—Diastatic Power Range 30-500 WK.

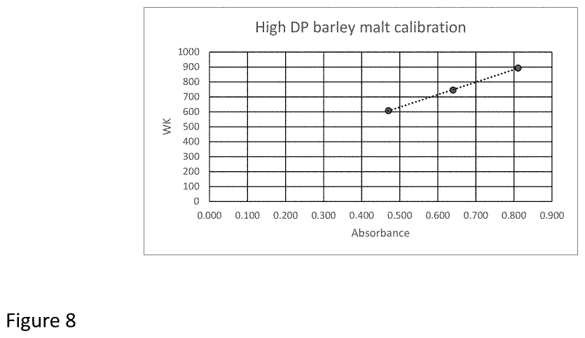

The test results obtained in example 2 and shown in FIG. 8 were applied as reference curve for barley malt.

The tests are repeated using different incubation times including the incubation times 3 minutes, 10 minutes and 20 minutes to thereby prepare additional sets of reference data correlated to respective incubation times as described above.

### Example 4

Ring Trial Validation—Incubation Time

Ring trial validations using three different milled malt samples having different WK values were performed.

The samples were tested according to the protocol given in example 2, where the buffer, the substrate, the stopping agent and the reading out were as in example 2.

Each sample was tested with three different incubation times, namely 3 minutes, 5 minutes and 7 minutes respectively.

Each test was performed in triplicate and the average absorbance parameter values were determined.

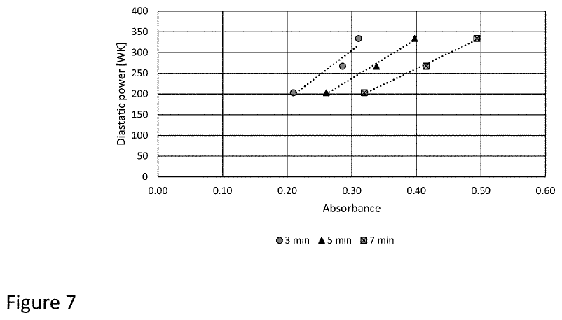

Thereafter the respective absorbance parameter values were plotted as a function of the Ring trial WK values. The plot is shown in FIG. 7.

The results show a very good correlation with the ring trial samples for each of the three incubation times.

Thus, by applying data pairs of the WK-values/determined absorbance parameter values as reference data, the computer system may be programmed to determine the WK value for an unknown sample tested according to the protocol and with an incubation time which may be any time within a range 3-7 minutes and at least some minutes beyond the 3-7 minutes incubation time.

### Example 5

Preparing Reference Curve for Barley Malt—Diastatic Power Range 500-100 WK.

Three samples of milled barley malt with different and known WK value were tested:

| Sample | Sample 1 | Sample 2 | Sample 3 |
|---|---|---|---|
| WK | 893* | 608 | 746 |

The samples were tested according to the protocol given in example 2, where the buffer, the substrate, the incubation time, the stopping agent and the reading out were as in example 2.
The actual WK value of the sample 1 material was 595, however, for test of this sample 50% more barley malt (i.e. 300 mg) was used to represent the 895 WK value.

Each test was performed in triplicate and the average absorbance parameter values were determined.

Thereafter the respective absorbance parameter values were plotted as a function of the Ring trial WK values. The plot is shown in FIG. 8.

The tests may be repeated using different incubation times. For providing two or more sets of reference data correlated to respective incubation times as described above.

### Example 6

Preparing Reference Curve for Wheat Malt—Diastatic Power Range 200-500 WK.

Four samples of milled wheat malt with different and known WK value were tested:

| Sample | Sample 1 | Sample 2 | Sample 3 | Sample 4 |
|---|---|---|---|---|
| WK | 223 | 269 | 390 | 445 |

The samples were tested according to the protocol given in example 2, where the substrate, the incubation time, the stopping agent and the reading out were as in example 2.

The extraction buffer was as in example 2 but the amount applied was 50 mL together with 70 mg Dithiothreitol/sample.

Each test was performed in triplicate and the average absorbance parameter values were determined.

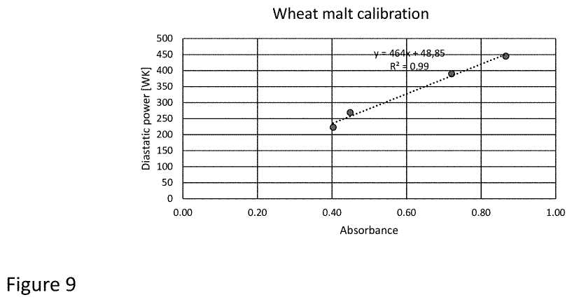

Thereafter the respective absorbance parameter values were plotted as a function of the Ring trial WK values. The plot is shown in FIG. 9.

The tests may be repeated using different incubation times. For providing two or more sets of reference data correlated to respective incubation times as described above.

### Example 7

Preparing Reference Curve for Alpha Amylase Activity for Barley Malt (10-100 DU)

Three different samples of barley malt with known DU values were subjected to an extraction according to the following extraction protocol:
Weigh 200 mg of milled malt.
Extraction with extraction buffer for 10 minutes at 35° C.
Syringe filtration with 0.45 μm filter. Collect the filtrate.

The three samples were subjected to a dilution series to provide in total 9 diluted samples, three from each of the samples A, B and C as listed below:

| | | Dilution ratio (always | |
|---|---|---|---|
| | mL buffer 3 - | pipette 250 μL of the | Known and |
| Sample | dilution step | extraction) | calculated-DU |
| Sample A | 45 | 181 | 10.4 |
| 3 - 51 | 9 | 37 | 51 |
| DU | 4.5 | 19 | 99.3 |
| Sample B | 36 | 145 | 10.2 |
| 6 - 40 | 12 | 49 | 30.2 |
| DU | 9 | 37 | 40.0 |
| Sample C | 27 | 109 | 22.3 |
| 10 - 65.6 | 9 | 37 | 65.6 |
| DU | 7 | 29 | 83.7 |

The samples were tested according to the following protocol:
Incubation of 250 μL filtrate with substrate for an incubation time at 35° C.
Stop the enzymatic cleaving by adding a stopping reagent
Read the resulting liquid at 410 nm in a spectrophotometer.

The buffer used for the dilution series was the maleic acid based extraction buffer described in example 1.

The substrate used was a glucose-based oligosaccharide conjugated with a chromophore, able to absorb visible light at 410 nm when the pH is higher than 9, mixed with a glucosidase. By the action of the enzyme of interest, in this case alpha amylase, the oligosaccharide is then accessible to the glucosidase, which then makes the chromophore free of glucose molecules. Such chromophore would then change color by the addition of the stopping reagent, and its intensity will depend directly on how much the enzyme of interest cleaves the oligosaccharide.

The incubation time used was 7 minutes.

The stopping agent used was an alkaline sodium carbonate solution.

The spectrophotometer used was the spectrometer sold by Glycospot under the trade name SIRIUS™.

The absorbance parameter for each sample was determined by withdrawing the light intensity transmitted through a blind from the light intensity transmitted through the respective samples.

Each test was performed in triplicate and the average absorbance parameter values were determined.

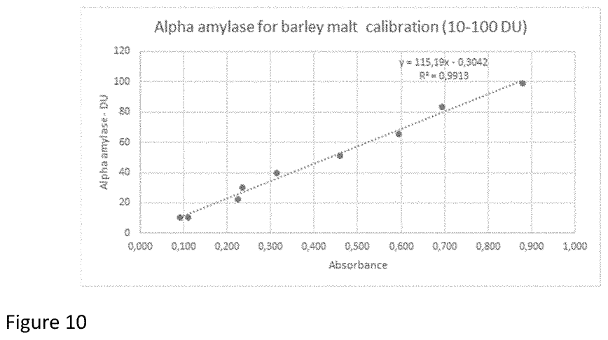

Thereafter the respective absorbance parameter values were plotted as a function of the DU values. The plot is shown in FIG. 10.

The tests may be repeated using different incubation times. For providing two or more sets of reference data correlated to respective incubation times as described above.

### Example 8

Preparing Reference Curve for Beta Amylase Activity in Malt in Betamyl-3 Units BU3

Betamyl-3 unit is the typical unit for beta amylase activity.

A single sample of barley malt with known BU3 value was subjected to an extraction according to the following extraction protocol:
Weigh 200 mg of milled barley malt.
Extraction with extraction buffer for 10 minutes at 35° C.
Syringe filtration with 0.45 μm filter. Collect the filtrate.

The sample were subjected to a dilution series to provide in total 6 diluted samples.

Each of the 6 dilution samples was tested according to the following protocol:
Incubation of 250 μL filtrate with substrate for an incubation time at 35° C.
Stop the enzymatic cleaving by adding a stopping reagent
Read the resulting liquid at 410 nm in a spectrophotometer.

The buffer used for the dilution series was the maleic acid based extraction buffer described in example 1.

The substrate used was a glucose-based oligosaccharide conjugated with a chromophore, able to absorb visible light at 410 nm when the pH is higher than 9, mixed with a glucosidase. By the action of the enzyme of interest, in this case beta amylase, the oligosaccharide is then accessible to the glucosidase, which then makes the chromophore free of glucose molecules. Such chromophore would then change color by the addition of the stopping reagent, and its intensity will depend directly on how much the enzyme of interest cleaves the oligosaccharide.

The incubation time used was 7 minutes.

The stopping agent used was an alkaline sodium carbonate solution.

The spectrophotometer used was the spectrometer sold by Glycospot under the trade name SIRIUS™.

The absorbance parameter for each sample was determined by withdrawing the light intensity transmitted through a blind from the light intensity transmitted through the respective samples.

Each test was performed in triplicate and the average absorbance parameter values were determined.

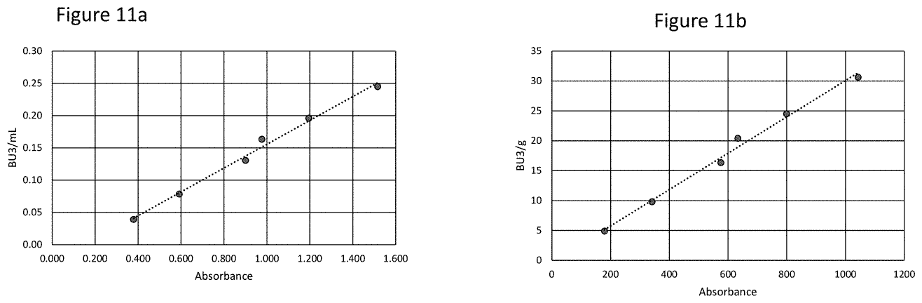

Thereafter the respective absorbance parameter values were plotted as a function of the BU3 values. The plot is shown in FIGS. 11a and 11b. FIG. 11a shows the respective absorbance parameter values versus the BU3/mL value and FIG. 11b shows the respective absorbance parameter values versus the BU3/g value.

The tests may be repeated using different incubation times. For providing two or more sets of reference data correlated to respective incubation times as described above.

### Example 9

Preparing Reference Curve for Beta Amylase Activity in Malt Extract

The test of example 8 was repeated but with the difference that on 1 g of milled barley malt was extracted using 25 mL extraction buffer and wherein the dilution series was providing 4 diluted samples.

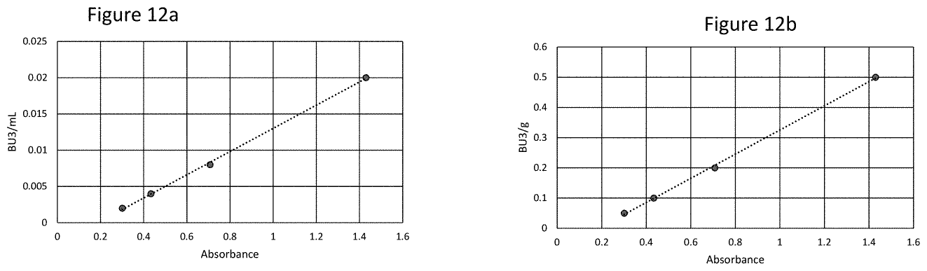

The respective absorbance parameter values were plotted as a function of the BU3 values. The plot is shown in FIGS. 12a and 12b. FIG. 12a shows the respective absorbance parameter values versus the BU3/mL value and FIG. 12b shows the respective absorbance parameter values versus the BU3/g value.

The tests may be repeated using different incubation times. For providing two or more sets of reference data correlated to respective incubation times as described above.

1. A method of determining activity of a target enzyme of a grain material, the method comprises
providing a fluid extract sample of the grain material using a preselected extraction procedure,
providing a dyed and/or chromogenic substrate for the target enzyme,
subjecting the fluid extract sample to the substrate for a preselected incubating time, and
determining the target enzyme activity of the grain material,
wherein the preselected extraction procedure comprises
milling the grain material to a predetermined particle size distribution,
preparing an extract from the milled substance by a method comprising thoroughly mixing a preselected amount of the milled grain material with a preselected amount of liquid extraction buffer and allowing the enzyme extraction for a preselected extraction time of up to about 20 minutes, and
taking the fluid extract sample of the liquid extraction buffer,
and wherein the step of determining the enzyme activity of the grain material comprises
determining an absorbance parameter of dye released from the substrate into the fluid extract sample and/or colored reaction product formed,
correlating the determined absorbance parameter to a standard curve associated to the preselected extraction procedure and representing absorbance parameter as a function of target enzyme activity.

2. The method of claim 1, wherein the step of milling the grain material comprises milling the grain material to a particle size where at least 90% by weight, such as at least 95% of the grain material passes a sieve of 0.5 mm (mesh 35) or less, such as, a sieve of 0.4 mm (mesh 40) or less, such as, a sieve of 0.3 mm (mesh 50) or less, such as, a sieve of 0.2 mm (mesh 70) or less.

3. The method of claim 1 or claim 2, wherein the step of milling the grain material comprises milling the grain material using a mill, such as a disc mill, a grist mill, a coffee mill and/or a Hammer mill.

4. The method of any one of the preceding claims, wherein the milling comprises milling a preselected amount of grain material for a preselected time.

5. The method of any one of the preceding claims, wherein the thoroughly mixing of a preselected amount of the milled grain material with a preselected amount of liquid extraction buffer comprises withdrawing at least about 10 mg, such as from about 0.1 g to about 5 g, such as from about 0.3 g to about 1 g of the milled grain material and mixing it with from about 5 ml to about 0.5 L per gram milled grain material, such as from about 10 ml to about 0.2 L per gram milled grain material.

6. The method of any one of the preceding claims, wherein the liquid extraction buffer is an aqueous buffer having a pH value of from about 4 to about 10, such as from about 5 to about 8.

7. The method of any one of the preceding claims, wherein the liquid extraction buffer comprises a reducing agent, such as Dithiothreitol (DTT) and/or tris(2-carboxyethyl)phosphine (TCEP).

8. The method of any one of the preceding claims, wherein the thoroughly mixing of a preselected amount of the milled grain material with a preselected amount of liquid extraction buffer comprises, shaking or stirring the mixture for a part of or for the entire extraction time.

9. The method of any one of the preceding claims, wherein the preselected extraction time is up to about 15 minutes, such as up to about 10 minutes, such as up to about 8 minutes, such as from about 2 to about 6 minutes.

10. The method of any one of the preceding claims, wherein the extraction is performed at a temperature from about 10 to about 40° C., such as from about 10 to about 30° C., such as from about 18 to about 24° C.

11. The method of any one of the preceding claims, wherein the preselected extraction time is terminated by taking the fluid extract sample of the liquid extraction buffer, preferably the fluid extract sample is essentially free of solids, such as free of particulate grain material.

12. The method of any one of the preceding claims, wherein the fluid extract sample is added to the substrate within 10 minutes from taking the fluid extract sample of the liquid extraction buffer, preferably within 5, such as within 2 minutes, such as immediately after taking the fluid extract sample of the liquid extraction buffer.

13. The method of any one of the preceding claims, wherein the preselected incubation time is up to about 30 minutes, such as from about 1 minute to about 20 minutes, the incubation time is preferably terminated by removing the non-digested substrate from the sample and/or by adding a stop agent.

14. The method of any one of the preceding claims, wherein the method comprises filtering of solid parts of the substrate prior to determining the absorbance parameter of dye released from the substrate into the fluid extract sample and/or colored reaction product formed, preferably the incubation time is terminated by filtering of solid parts of the substrate.

15. The method of any one of the preceding claims, wherein the determining of the target enzyme activity of the grain material comprises measuring the absorbance parameter of the fluid extract sample with released dye, wherein the absorbance parameter comprises at least one wavelength absorbable by the dye.

16. The method of any one of the preceding claims, wherein the standard curve further is associated to the selected dyed and/or chromogenic substrate and preselected incubation time.

17. The method of any one of the preceding claims, wherein the target enzyme is α-Amylase, β-Amylase, Limit Dextrinase, Beta-glucanase, lipase, cellulase, xylanase, protease or any combinations comprising one or more of these.

18. The method of any one of the preceding claims, wherein the grain material is selected from one or more of cereals, nuts, legumes, spices and any combinations comprising at least one of these.

19. The method of any one of the preceding claims, wherein the grain material is barley, wheat, rye or oat or any combinations comprising one or more of these.

20. The method of any one of the preceding claims, wherein the dyed and/or chromogenic substrate is specific for the target enzyme.

21. The method of any one of the preceding claims, wherein the selected dyed and/or chromogenic substrate is a gelled biopolymer substrate comprising cross-linked polymeric biomolecules selected from polynucleotides, polypeptides, polysaccharides or any combinations thereof, preferably the dyed and/or chromogenic substrate is an aerogel or a xerogel.

22. The method of any one of the preceding claims, wherein the method further comprises generating the standard curve, wherein the generation of the standard curve comprises
providing a plurality of grain material reference samples with different and known enzyme activity of the target enzyme,
providing a fluid extract reference sample of each of the respective grain material reference samples using a preselected reference extraction procedure,
providing a selected dyed and/or chromogenic reference substrate for the target enzyme for each of the fluid extract reference sample,
subjecting each of the respective fluid extract reference samples to the respective selected dyed and/or chromogenic reference substrates for a preselected reference incubation time,
determining an absorbance parameter of dye released from each of the respective reference substrates into the fluid extract sample and/or colored reaction product formed,
performing a regression including points of respective pairs of determined absorbance parameter and corresponding, known enzyme activity to provide a linear standard curve.

23. The method of claim 22, wherein the plurality of grain material reference samples with different and known enzyme activity of the target enzyme comprises at least 3 grain material reference samples, such as at least 5 grain material reference samples, such as at least 8 grain material reference samples.

24. The method of claim 22 or claim 23, wherein the selected dyed and/or chromogenic reference substrate are identical to the selected dyed and/or chromogenic substrate.

25. The method of claim 22 or claim 23, wherein the preselected incubation time for the fluid extract reference samples are from 5 minutes shorter to 5 minutes longer than the incubation time for the fluid extract samples, such as from 1 minute shorter to 1 minute longer, such as from 0.5 minutes shorter to 0.5 minutes longer.

26. A system for determining activity of a target enzyme of a grain material, the system comprises
an extraction container,
an extraction buffer for extracting the preselected target enzyme,
container, such as a filter vial with a corresponding independent filter piston or a microcentrifuge tube,
a selected dyed and/or chromogenic substrate for said preselected target enzyme,
a spectroscope comprising a light source and an optical reader and
a computer system,
wherein the computer system comprises a memory storing data representing a standard curve associated to a preselected reference extraction procedure for extracting target enzyme from a grain material and representing absorbance parameter as a function of target enzyme activity, wherein the preselected reference extraction procedure comprises
milling the grain material to a predetermined particle size determine by sieving,
preparing an extract from the milled substance by a method comprising thoroughly mixing a preselected amount of the milled grain material with a preselected amount of liquid extraction buffer and allowing the enzyme extraction for a preselected reference extraction time up to about 20 minutes, and
taking the fluid extract sample of the liquid extraction buffer.

27. The system of claim 26, wherein the standard curve further is associated to the selected dyed and/or chromogenic substrate and preselected incubation time.

28. The system of claim 26 or claim 27, wherein said spectroscope is arranged for determining an absorbance parameter of a sample in a sample container in reflecting mode and/or in transmitting mode.

29. The system of any one of claims 26-28, wherein said light source of said spectroscope is configured for emitting at least one wavelength absorbable by said dye.

30. The system of any one of claims 26-29, wherein said optical reader is configured for reading at least one wavelength absorbable by said dye.

31. The system of any one of claims 26-30, wherein said computer is configured for
receiving data from the spectroscope representing a determined absorbance parameter,
correlating the data representing the determined absorbance parameter to the data representing the standard curve and determine data representing the target enzyme activity, and
transmitting said data representing the target enzyme activity.

32. The system of any one of claims 26-31, wherein said computer is programmed for timely organizing an assay for determining of target enzyme activity comprising
receiving data representing a start incubation time,
transmitting data representing a stop incubation time after the preselected incubation time of receiving the start incubation time,
receiving data from the spectroscope representing a determined absorbance parameter,
correlating the data representing the determined absorbance parameter to the data representing the standard curve and determine data representing the target enzyme activity, and
transmitting said data representing the target enzyme activity.

33. The system of any one of claims 26-31, wherein said computer is programmed for timely organizing an assay for determining of target enzyme activity comprising
receiving data representing a start extraction time,
transmitting data representing a stop extraction time after the preselected extraction time of receiving the start extraction time,
receiving data representing a start incubation time,
transmitting data representing a stop incubation time after the preselected incubation time of receiving the start incubation time,
receiving data from the optical reader representing a determined absorbance parameter,
correlating the data representing the determined absorbance parameter to the data representing the standard curve and determine data representing the target enzyme activity, and
transmitting said data representing the target enzyme activity.

34. The system of any one of claims 26-33, wherein said computer is programmed for timely organizing an assay according to the method of any one of claims 1-25.

35. The system of any one of claims 26-34, wherein said memory of the computer stores data representing two or more standard curves, each associated to respective preselected extraction procedures for extracting target enzyme from a grain material and representing absorbance parameter as a function of target enzyme activity, wherein the respective preselected extraction procedures for extracting target enzyme from a grain material differs from each other with respect to preselected reference extraction time.

36. The system of claim 35, wherein the computer is configured for receiving data representing a start extraction time,
receiving data representing a stop extraction time,
determining the extraction time,
correlating the extraction time to the two or more standard curves and determining a best fit standard curve,
correlating the data representing the determined absorbance parameter to the data representing the best fit standard curve and determine data representing the target enzyme activity, and
transmitting said data representing the target enzyme activity.

37. The system of claim 36, wherein the best fit standard curve is a calculated best fit standard curve obtained by performing a regression between two or more of said standard curves stored on said memory.

38. The system of any one of claims 26-37, wherein said memory of the computer stores data representing two or more standard curves, each associated to respective preselected reference incubation procedures for digestion a substrate by a fluid extract sample and representing absorbance parameter as a function of target enzyme activity, wherein the respective preselected reference incubation procedures for digestion a substrate by a fluid extract sample differs from each other with respect to preselected reference incubation time.

39. The system of claim 38, wherein the computer is configured for
receiving data representing a start incubation time,
receiving data representing a stop incubation time,
determining the incubation time,
correlating the incubation time to the two or more standard curves and determining a best fit standard curve,
correlating the data representing the determined absorbance parameter to the data representing the best fit standard curve and determine data representing the target enzyme activity, and
transmitting said data representing the target enzyme activity.

40. The system of claim 39, wherein the best fit standard curve is a calculated best fit standard curve obtained by performing a regression between two or more of said standard curves stored on said memory.
